(19) United States
(12) Patent Application Publication
(10) Pub. No.: US 2024/0407831 A1
(43) Pub. Date: Dec. 12, 2024

(54) SILICONE CONSTRUCTION VESSEL SEALING DEVICE

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Theodore C. Blus, Arden Hills, MN (US); John Mensch, Plymouth, MN (US)

(21) Appl. No.: 18/699,935

(22) PCT Filed: Oct. 21, 2022
(86) PCT No.: PCT/US2022/078481

§ 371 Date: Apr. 10, 2024

Related U.S. Application Data
(60) Provisional application No. 63/270,707, filed on Oct. 22, 2021.

(51) Int. Cl. A61B 18/14; A61B 18/00

(52) U.S. Cl. CPC A61B 18/1445; A61B 2018/00077; A61B 2018/00083; A61B 2018/00136; A61B 2018/00148

(57) ABSTRACT

A forceps is provided that includes a first frame and a second frame opposing the first frame. One or both of the first and second frames include a polymer overmold where each of the first and second polymer overmolds have first and second conductor layers deposited thereon. The first and second conductor layers can have the same pattern or alternating patterns. When the first and second conductor layers have the same pattern, the forceps can include standoffs such that the first and second conductor layers are electrically isolated when the forceps is in a closed position. When the first and second conductor layers have alternating patterns, when the forceps is in a closed position, the patterns alternate such that the first and second conductor layers remain electrically isolated.

### CROSS-REFERENCE TO RELATED APPLICATION

This application claims the benefit of priority to U.S. Provisional Patent Application Ser. No. 63/270,707, filed Oct. 22, 2021, the contents of which are incorporated herein by reference in their entirety.

### TECHNICAL FIELD

This document pertains generally, but not by way of limitation, to surgical devices that can be used for various surgical procedures. More specifically, but not by way of limitation, the present application relates to an electrosurgical forceps.

### BACKGROUND

Laparoscopic surgery is a minimally invasive procedure performed in the abdomen or pelvis. Laparoscopic surgery uses small incisions at the abdomen, which can reduce hemorrhaging and reduce recovery time typically associated with surgical procedures. Laparoscopic forceps are used during laparoscopic surgeries in order to grasp, desiccate, seal, coagulate and cut tissue. Laparoscopic forceps can grasp tissue and move the tissue in order to allow a medical practitioner to perform a medical procedure in small surgical fields.

In order to have the ability to grasp, coagulate, and move tissue in small spaces, a laparoscopic forceps is small in size where the parts that form the laparoscopic forceps require tight tolerance constraints due to the small size of the laparoscopic forceps. Typically, a laparoscopic forceps includes a number of parts that provide grasping and electric cauterization functionality. However, the tight tolerances require precision in manufacturing the parts and assembling the parts, thereby increasing cost and the possibility of fabricating a laparoscopic forceps unsuitable for its intended purpose.

Furthermore, a surface of a laparoscopic forceps includes a material that allows for electric cauterization, such as a metallic surface. However, when laparoscopic forceps having a metallic surface grasp tissue, the tissue can slip out of the laparoscopic forceps due to the innately slippery nature of a metallic surface.

### SUMMARY

Accordingly, what is needed is a laparoscopic forceps that is formed of fewer components and having a tissue surface capable of firmly grasping and holding tissue in place.

Examples solve the problems noted above by providing a laparoscopic forceps that is formed from fewer components and in some examples, having a pliable surface that is capable of firmly grasping and holding tissue in place. A laparoscopic forceps includes a first frame and a second frame where at least one of, or each of the first frame and the second frame have a silicone overmold formed thereon. The silicone overmolds can be injection molded and then respectively placed on the first frame and the second frame. Each of the silicone overmolds can include a surface that contacts tissue during use of the laparoscopic forceps. By virtue of being formed of silicone, the silicone overmolds can be pliable and firmly grasp and hold tissue in place during use of the laparoscopic forceps. The laparoscopic forceps can also include a deposited conductor layer formed on a surface of the silicone overmold (e.g., at the tissue sealing surface). The deposited conductor layer can include a molecularly conductive ink that that is screen printed on to a surface of the silicone overmold.

### BRIEF DESCRIPTION OF FIGURES

FIG. 1 illustrates a laparoscopic forceps in accordance with at least one example of the present disclosure.

FIG. 2 illustrates jaws of the laparoscopic forceps of FIG. 1 in greater detail in accordance with at least one example of the present disclosure.

FIGS. 3A and 3B show a configuration of a conductor layer of the jaws of FIG. 2 in accordance with at least one example of the present disclosure.

FIG. 4 shows an alternative configuration of a conductor layer of the jaws of FIG. 2 in accordance with at least one example of the present disclosure.

FIGS. 5A-5C show an alternative configuration of a conductor layer of the jaws of FIG. 2 in accordance with at least one example of the present disclosure.

FIG. 6 illustrates a method of forming the jaws of FIG. 2 in accordance with at least one example of the present disclosure.

FIGS. 7 and 8 show an alternative configuration of a conductor layer of the jaws of FIG. 2 in accordance with at least one example of the present disclosure.

FIG. 9 is a flowchart indicating a reprocessing method of the jaws of FIG. 2 in accordance with at least one example of the present disclosure.

### DETAILED DESCRIPTION

Examples of the present disclosure relate to a laparoscopic forceps includes a first frame and a second frame where at least one of, or each of the first frame and the second frame have a silicone (or other polymer) overmold formed thereon. The silicone overmold(s) can be injection molded and then respectively placed on the first frame and the second frame, or can be directly overmolded onto the first and second frames. Each of the silicone overmolds can include a surface that contacts tissue during use of the laparoscopic forceps. By virtue of being formed of silicone, the silicone overmolds can be pliable and firmly grasp and hold tissue in place during use of the laparoscopic forceps. The laparoscopic forceps can also include a deposited conductor layer formed on a surface of the silicone overmold formed by various suitable methods. For example, the deposited conductor layer can include a molecularly conductive ink that that is screen printed on to a surface of the silicone overmold.

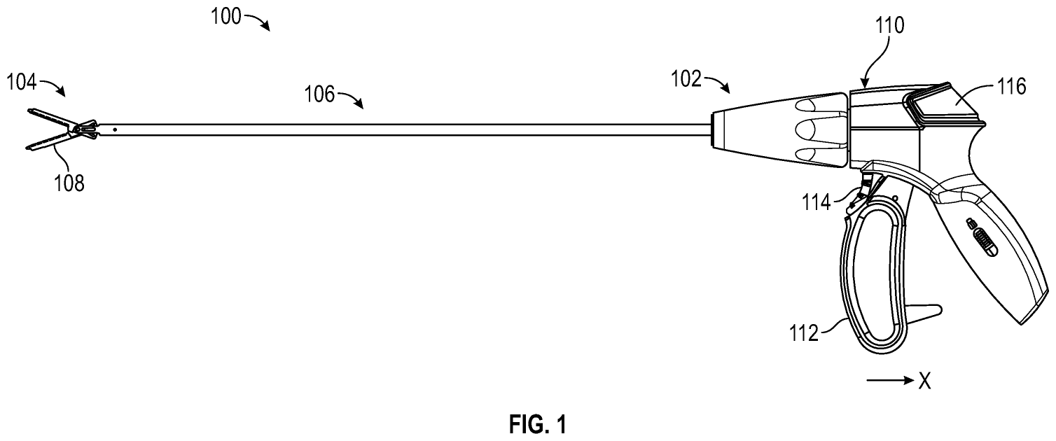

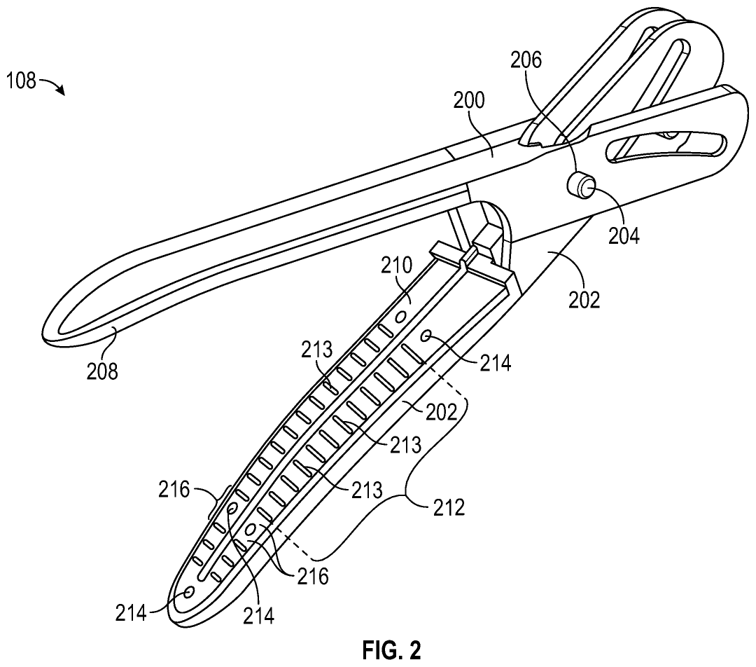

Now making reference to FIG. 1, a forceps 100 is shown. The forceps 100, which can be used for any type of electrosurgical surgical, including but not limited to laparoscopic procedures, can include a handpiece 102 at a proximal end and an end effector 104 at a distal end. An intermediate portion 106 can extend between the handpiece 102 and the end effector 104 to operably couple the handpiece 102 to the end effector 104. Various movements of the end effector 104 can be controlled by one or more actuation systems of the handpiece 102. The end effector 104 can include forceps jaw 108 that are capable of opening and closing. The end effector 104 can include a cutting blade (not shown) and a conductive layer 212 (FIG. 2) for applying electromagnetic energy to tissue grasped by the forceps jaw 108. The forceps 100 can also include a housing 110, a lever 112, a trigger 114 and an activation button 116. The end effector 104, or a portion of the end effector 104, can be one or more of: opened, closed, rotated, extended, retracted, and electromagnetically energized (e.g., electrically energized). In some examples, the energy can be radio-frequency energy applied at the tissue sealing surfaces of the opposing jaw faces.

To operate the end effector 104, a user can displace the lever 112 proximally by applying a force along a direction X to drive the forceps jaw 108 between an open position and a closed position. Moving the forceps jaw 108 from the open position to the closed position allows a user to clamp down on and grasp tissue. When the forceps jaw 108 clamp tissue, a user can depress the activation button 116 to cause an electromagnetic energy, or in some examples, ultrasound, to be delivered to the end effector 104, such as to the conductive layer 212 (FIG. 2). Application of electromagnetic energy can be used to seal or otherwise affect the tissue being clamped. The electromagnetic energy can cause tissue to be coagulated, cauterized, sealed, ablated, desiccated or can cause controlled necrosis.

Making reference to FIG. 2, the forceps jaw 108 can include a first forceps jaw frame 200 that is operatively coupled to a second forceps jaw frame 202 via a coupling 204, such as a pivot. The frame coupling 204 can pass through a hole 206 of the first forceps jaw frame 200 and another hole within the second frame (not shown) thereby operatively coupling the first forceps jaw frame 200 with the second forceps jaw frame 202. The first forceps jaw frame 200 and the second forceps jaw frame 202 can be operatively coupled such that the first forceps jaw frame 200 and the second forceps jaw frame 202 rotate relative to each other such to allow the forceps jaw 108 to have an open position as shown in FIG. 2 and a closed position (not shown). The frame coupling can be a pin, a dowel, or any other type of coupling that allows for rotation of the first forceps jaw frame 200 relative to the second forceps jaw frame 202. In some examples the jaw frame can be formed of a metal, such as stainless steel, aluminum, titanium, a carbon fiber or other suitable structural material.

The forceps jaw 108 can also include a first overmold 208 located on the first forceps jaw frame 200 along with a second overmold 210 located on the second forceps jaw frame 202. The first and second overmolds 208 and 210 can be formed of any type of material that is capable of electrically insulating the first and second forceps jaw frames 200 and 202. Examples of materials that can be used can include any suitable biocompatible and sufficiently electrically insulative material including but not limited to: silicone, polysiloxane, a polymer such as pliable plastics, silica-reinforced polymers, silsesquioxanes, and POSS-based polymers, glass, nylon, glass filled nylon, ceramic, or the like. When the first and second overmolds 208 and 210 are formed of a pliable material, such as silicone to form a silicone overmold, by virtue of the pliable material, the resulting silicone overmold is capable of gripping tissue clasped by the forceps jaw 108. More specifically, the overmolds 208 and 210 can deform around the tissue or any other material or object clasped by the forceps jaw 108. Each of the first and second overmolds 208 and 210 can be injected molded and then respectively placed onto the first and second forceps jaw frames 200 and 202 to have the configuration shown with reference to FIG. 2.

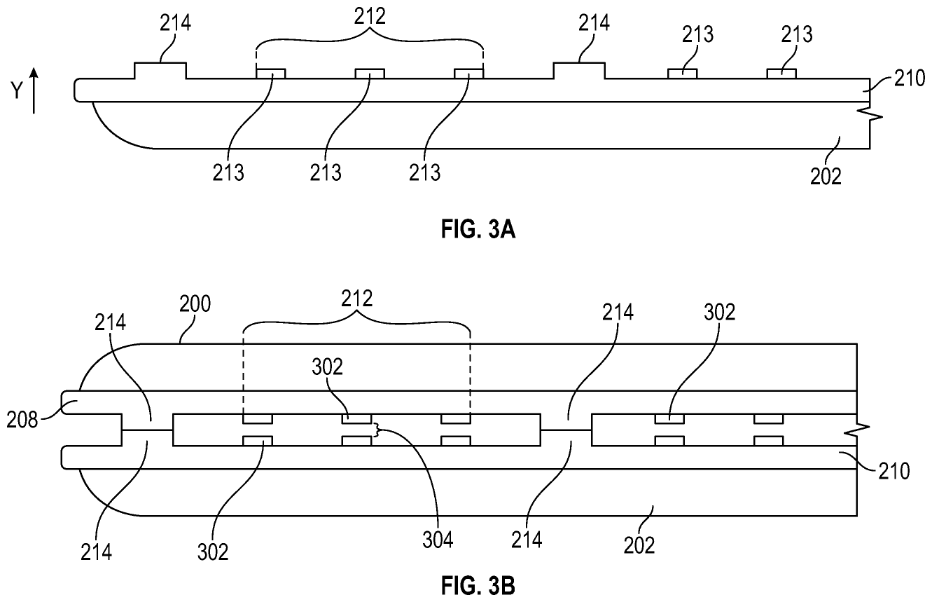

The forceps jaw 108 can also include a conductor layer 212 defined by conductor layer traces 213 disposed on a surface 300 (FIG. 3A) of the first or second overmolds 208 or 210. While the forceps jaw 108 are shown as having only one conductor layer deposited on the second overmold 210, the first overmold 208 can also have a conductor layer having patterns and characteristics similar to the conductor layer 212 as discussed herein where the conductor layer of the first overmold 208 opposes the conductor layer 212 of the second overmold 210. The conductor layer traces 213 can be deposited to create the conductor layer 212 on the second overmold surface 300 via any deposition technique. Techniques can include using a printing device where the printing device uses a pen to deposit the conductor layer traces 213 onto the second overmold surface 300 thereby creating the conductor layer 212. The conductor layer traces 213 can also be screen printed onto the overmold surface 300 using any suitable screen printing process to create the conductor layer 212. In addition, the conductor layer traces 213 can be formed on the second overmold surface 300 using any suitable additive manufacturing technique to create the conductor layer 212. In some examples, the conductive ink can be 3-dimensionally (3D) printed on to the overmold. In some examples, the deposited conductor layer can be applied by chemical vapor deposition (CVD), including but not limited to plasma-enhanced chemical vapor deposition (PECVD). While some examples described herein reference the application of a molecularly conductive ink, any of the suitable manufacturing methods can be used to apply the conductor layers described herein. All combinations of methods and conductor layer arrangements are considered within the scope of this disclosure.

The conductor layer 212 can be an electrical conductor layer formed of a deposit such as an electrically conductive ink, such as a molecularly conductive ink, that is patterned on to the second overmold surface using the techniques described above. The molecularly conductive ink can be any type of ink that conducts electricity. Examples of molecularly conductive ink can include ink infused with graphite, graphine, or carbon nanotubes, silver ink, or the like. The conductive layer 212 can apply via the conductor layer traces 213 electromagnetic energy to tissue being clasped by the forceps jaw 108 to achieve the results discussed herein during electromagnetic energy application, such as to seal tissue. The molecularly conductive ink can be patterned on to the second overmold surface 300 to create the conductor layer traces 213 on the conductor layer 212.

The forceps jaw 108 can also include standoffs 214 located on the second overmold 210. While the forceps jaw 108 are shown as having the standoffs 214 formed on the second overmold 210, in some examples, the first overmold 208 can also have the standoffs 214, as shown in FIG. 3B. As can be seen with reference to FIG. 3A, the standoffs 214 project/protrude from the second overmold surface 300 and beyond the second overmold surface 300 along a direction Y a greater distance than the conductor layer traces 213. By virtue of projecting further along the direction Y, the standoffs 214 can function to maintain a gap 304 between the conductor layer traces 213 when the forceps jaw 108 are in a closed position, as shown in FIG. 3B. In particular, the standoffs 214 on the first overmold 208 align with the standoffs 214 on the second overmold 210 such that the standoffs 214 on the first overmold 208 abut and rest on the standoffs 214 of the second overmold 210. As a result of the standoffs 214 maintaining the gap 304, the conductor layer traces 213 at the first overmold 208 are electrically insulated from the conductor layer traces 213 at the second overmold 210 such that electromagnetic energy provided by the conductor layer traces 213 is provided to tissue clasped by the forceps jaw 108 instead of conducting through the conductor layer traces 213 and bypassing clasped tissue.

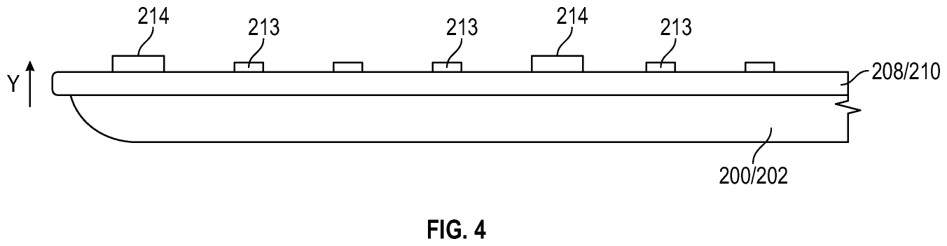

The standoffs 214 can be formed as part of the first and second overmolds 208 and 210 as shown with reference to FIGS. 3A and 3B. Here, when the first and second overmolds 208 and 210 are formed, such as with an injection molding process, the standoffs 214 can also be formed such that the standoffs 214 are unitary, e.g., form a single piece, with the first and second overmolds 208 and 210. Moreover, the standoffs 214 can be separately formed onto a surface of each of the first and second overmolds 208 and 210, as shown in FIG. 4. While only the second overmold 210 is shown as having the standoffs 214 formed on the second overmold surface 300, the first overmold surface 208 can have standoffs having the same configuration or a different configuration as shown with reference to FIG. 4 and described herein. In FIG. 4, after the second overmold 210 is formed, the standoffs 214 can be formed onto the second overmold surface 300 using any suitable technique. The standoffs 214 can be formed from a non-conductive material, such as ceramic, glass, plastic, or the like. Additionally, the standoffs 214 can be formed to project along the direction Y further away from the second overmold surface 300 in comparison to the conductor layer traces 213, as can be seen in FIG. 4.

As noted above, the conductor layer traces 213 can be patterned onto the first and second overmolds 208 and 210 to form the conductor layer 212. In examples where the first and second overmolds 208 and 210 include the standoffs 214, molecularly conductive ink that is deposited onto the first and second overmolds 208 and 210 to create the conductor layer traces 213 and the conductor layer 212 can be deposited to create a pattern that creates voids (e.g., opening) around the standoffs 214 or around individual ones of the standoffs 214. To further illustrate, the conductor layer 212 can include voids 216 as shown in FIG. 2 where no conductor layer traces 213 are formed. Thus, when the standoffs 214 contact and abut each other, no conductor layer traces 213 can inadvertently contact each other thereby allowing electrical conduction to bypass grasped tissue.

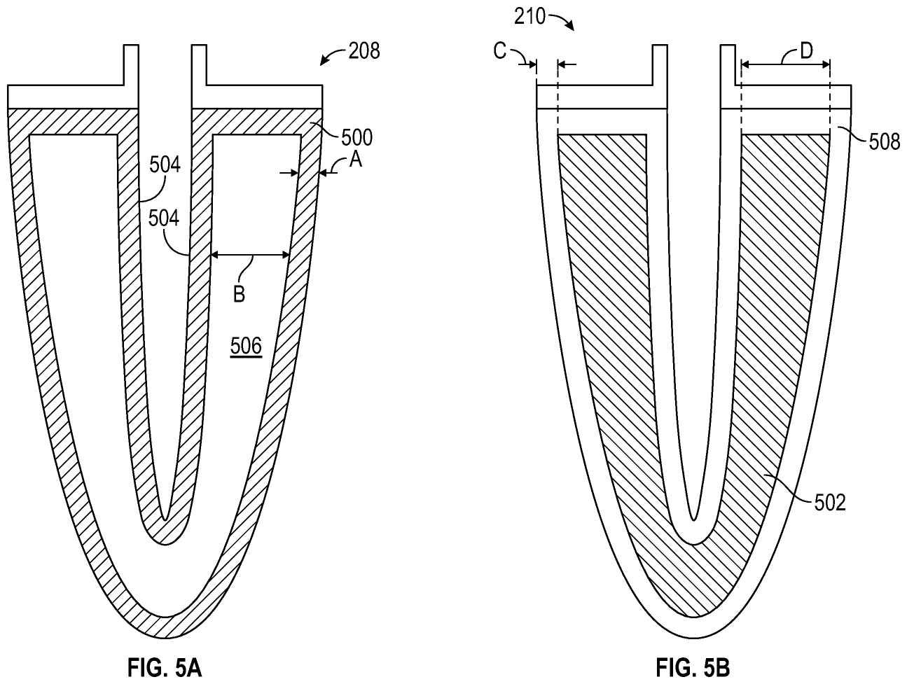

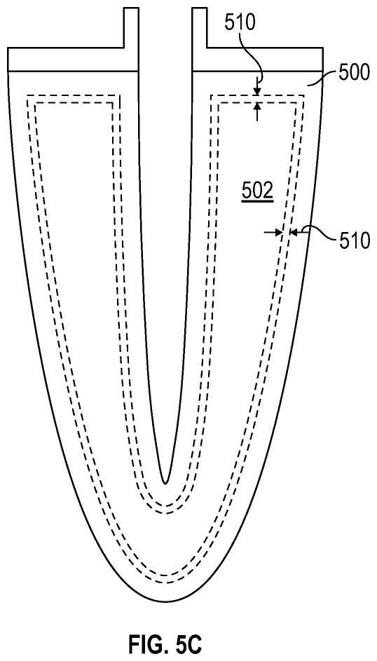

In addition to employing the standoffs 214 to maintain the gap 304 and thereby prevent the conductor layer traces 213 on the first overmold 208 from contacting the conductor layer traces 213 on the second overmold 210, a conductor layer trace can have a first pattern on the first overmold 208 and a second pattern on the second overmold 210, as show with reference to FIGS. 5A and 5B. Here, the first overmold 208 has a first conductor layer trace pattern 500 that can correspond to a first footprint while the second overmold 210 has a second conductor layer trace pattern 502 that can correspond to a second footprint. The first conductor layer trace pattern 500 is different from the second conductor layer trace pattern 502.

As may be seen with reference to FIG. 5A, the first conductor layer trace pattern 500 can alternate with the second conductor layer trace pattern 502 when the forceps jaw 108 are in a closed position. More specifically, the first conductor layer trace pattern 500 can be formed adjacent an outer periphery 504 and have a width A. Furthermore, the first conductor layer trace pattern 500 is formed such that an inner area 506 of the first overmold 208 has a width B and does not include any molecularly conductive ink.

The second conductor layer trace pattern 502 on the second overmold 210 has a pattern that alternates with the first conductor layer trace pattern 500. In particular, an outer area 508 of the second overmold 210 does not include the molecularly conductive ink. Instead, the second conductor layer trace pattern 502 is located at an inner area of the second overmold 210, as shown in FIG. 5B. The outer area 508 can have a width C that is slightly larger than the width A. Furthermore, the second conductor layer trace pattern 502 can be formed to a width D that is slightly smaller than the width B. As such, when the forceps jaw 108 close, as shown in FIG. 5C, a gap 510 (shown in FIG. 5C) can be formed between the first conductor layer trace pattern 500 and the second conductor layer trace pattern 502 such that the first and second conductor layer trace patterns 500 and 502 can be electrically isolated from each other. In particular, the first print of the first conductor layer trace pattern 500 does not overlap, i.e., is non-overlapping with respect to, the second footprint of the second conductor layer trace pattern 502. Each of the first and second conductor layer trace patterns 500 and 502 can be formed with the molecularly conductive ink using the techniques previously discussed. Additionally, the molecularly conductive ink used to form the first and second conductor layer trace patterns 500 and 502 can have the properties discussed above.

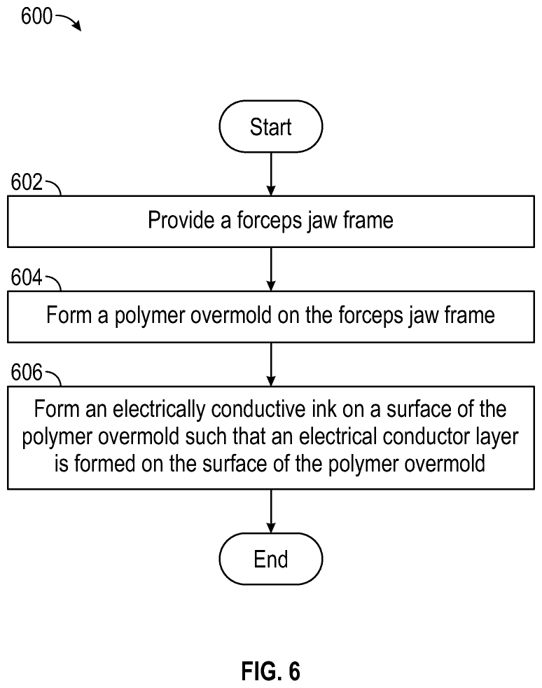

Now making reference to FIG. 6, a method 600 of forming a forceps is described. Initially, during an operation 600, a forceps jaw frame for the forceps is provided. The frame can include a first frame and a second frame that will oppose the first frame when the forceps is completed. After providing the frame, the method 600 performs an operation 604 where a polymer overmold is formed on the forceps jaw frame. In particular, a first overmold can be formed on the first frame and a second overmold can be formed on the second frame such that the first overmold opposes the second overmold.

As an example of the method 600, during the operation 602, a first frame such as the first forceps jaw frame 200, and a second frame, such as the second forceps jaw frame 202, can be provided. The first and second forceps jaw frames 200 and 202 can be formed using any suitable techniques. During the operation 604, the first and second overmolds 208 and 210 can be formed with an injection molding process where, during formation, the standoffs 214 can also be formed as part of the first and second overmolds 208 and 210. In addition, during the operation 604, the first overmold 208 can be placed or molded onto the first forceps jaw frame 200 and the second overmold 210 can be placed or molded onto the second forceps jaw frame 202.

After completion of the operation 604, the method 600 can perform the operation 606, where a electrically conductive ink (or depositing of any other conductive layer by any method described herein) is formed onto a surface of the silicone overmold. The molecularly conductive ink can be deposited onto the silicone overmold such that a electrical conductor layer is formed on the surface of the silicone overmold having the properties and functionality previously discussed. The molecularly conductive ink can be deposited onto a first overmold and a second overmold to from first and second conductor layer trace patterns. Furthermore, the molecularly conductive ink can be deposited such that the first conductor layer trace pattern is electrically isolated from the second conductor layer trace pattern. The molecularly conductive ink can be deposited to form voids for standoffs. Alternatively, the molecularly conductive ink can be deposited such that a first conductor layer trace pattern is formed on the first overmold that alternates with a second conductor layer trace pattern on the second overmold as discussed above. In some examples, the conductive layer can be formed on the overmold prior to forming the overmold onto the frame. More specifically, after the overmold is formed, the conductor layer can be formed on a surface of the overmold. Once the conductor layer is formed on the overmold, the overmold can be formed onto frame.

Returning to the example, during the operation 606, a molecularly conductive ink can be deposited onto the surface 300 of the first overmold 208 such that the first conductor layer trace pattern 500 is formed. Moreover, during the operation 606, a molecularly conductive ink can be deposited onto the surface 300 of the second overmold 210 such that the second conductor layer trace pattern 502 is formed. Any number of conductor layer patterns can be deposited to obtain the desired number and arrangement of traces on the silicone overmold. Upon completion of the operation 606, the method 600 is complete.

The laparoscopic forceps in accordance with the above can comprise three components, a frame, a silicone overmold, and a conductor layer formed on a surface of the silicone overmold. Since the laparoscopic forceps only includes two principal components, the frame and the overmold with the conductor layer formed thereon, the problems discussed above are avoided. In particular, the complexity associated with manufacturing the laparoscopic forceps is reduced since fewer tolerance constraints need to be met. In addition, the overmold can be formed of a pliable material such as silicone. The pliable material can firmly grip tissue clamped by the laparoscopic forceps, thereby decreasing the possibility of the clamped tissue slipping out of the laparoscopic forceps.

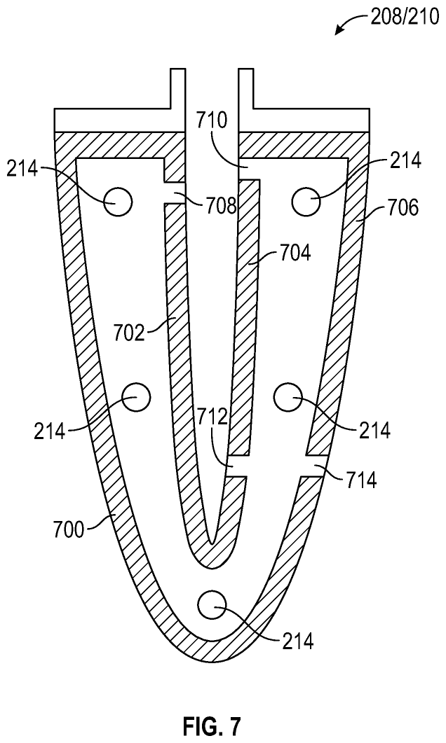

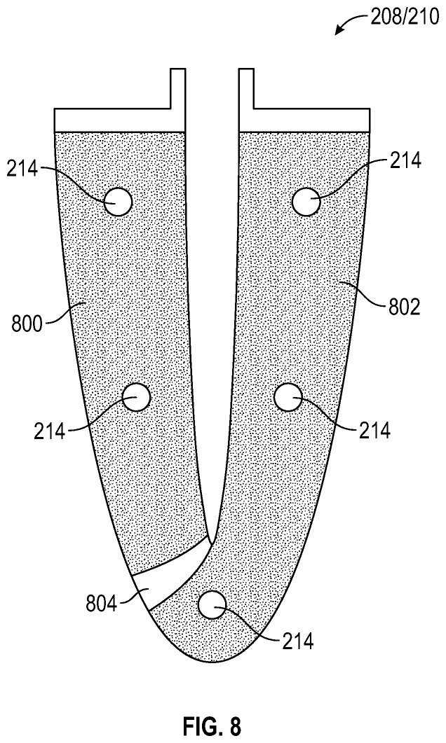

Examples can also include a combination of the standoffs 214 and conductor layer trace patterns, as shown with reference to FIGS. 7 and 8. In particular, the first overmold 208 or the second overmold 210 can include conductor layer trace patterns 700-706 separated by gaps 708-714. The conductor layer trace patterns 700-706 can be formed using the techniques described herein. Moreover, the conductor layer trace pattern 700-706 can have either a negative or positive polarity. The gap 708 can separate the conductor layer trace patterns 700 and 702. The gap 710 can separate the conductor layer trace patterns 704 and 706. The gap 712 can separate the conductor layer trace patterns 702 and 704. The gap 714 can separate the conductor layer trace patterns 700 and 706. Thus, the gaps 708-714 can electrically isolate the conductor layer trace patterns 700-706.

Referring to FIG. 8, the first overmold 208 or the second overmold 210 can include conductor layer trace patterns 800 and 802 separated by a gap 804. The conductor layer trace patterns 800 and 802 can be formed using the techniques described herein. Moreover, the conductor layer trace pattern 800 and 802 can have either a negative or positive polarity. The gap 804 can separate the conductor layer trace patterns 800 and 802.

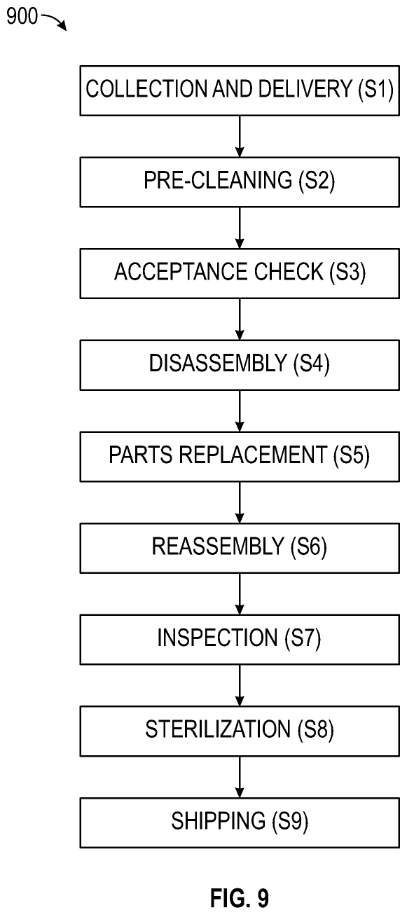

FIG. 9 is a flowchart indicating a reprocessing method 900 for any of the forceps or jaws described herein, and will be described generally as the forceps 100. FIG. 9 is a flowchart indicating the reprocessing method for the forceps 100. The forceps 100 described above may be disposed of after one use, or may be repeatedly used a plurality of times. In the case of a configuration that is repeatedly used a plurality of times, for example, reprocessing method shown in FIG. 9 can be used or may be required. The reprocessing methods described herein can be used with any of the forceps described herein, although other reprocessing methods may also be used with any of the forceps described herein.

An operator who remanufactures devices collects the used forceps 100 after the forceps 100 has been used for treatment. The operator can transport the forceps 100 to a factory or the like (Step S1). At this time, the used forceps 100 is transported in a dedicated container to prevent contamination of the forceps 100.

Then, the operator cleans and sterilizes the collected and transported used forceps 100 (Step S2). Specifically, in cleaning the forceps 100, deposits adhering to the jaws of the forceps 100 are removed by using a brush or the like. After that, to remove pathogenic microorganisms and the like derived from blood, body fluid, etc., a cleaning solution such as an isopropanol-containing cleaning agent, proteolytic enzyme detergent, and alcohol is applied and the jaws in order to further clean the forceps 100. The cleaning liquid is not limited to the cleaning liquid described above, and other cleaning liquids may be used. Further, in the sterilization of the forceps 100, to sterilize the pathogenic microorganisms and the like adhering to the jaws, any of high-pressure steam sterilization, ethylene oxide gas sterilization, gamma ray sterilization, hydrogen peroxide and hydrogen peroxide low temperature sterilization can be used. The jaws are reusable and may be easy to clean.

The operator performs an acceptance check of the used forceps 100 (Step S3). In detail, the operator checks whether the used forceps 100 has significant defects or the used forceps 100 exceed a maximum number of reprocessing. In particular, the conductor layer, the silicone overmold and exposed portions of the jaw frame may be examined.

Next, the operator disassembles or removes portions of the used forceps 100 to be replaced (Step S4). Specifically, if the conductor layer is damaged, at least a portion of the conductor layer can be removed from the jaws, such as by sand blasting, chemical etching or chemical removal, or temperature treatment to separate the conductor layer from the silicone overmold.

After the step S4, a new conductor layer can be applied during a step S5. If it is difficult to access the jaws, the jaws could be separated or at least partially separated from the forceps to provide better access to apply the conductor layer.

After step S5, the operator assembles a new forceps 100, if required. (Step S6).

In some examples, Step S6 can include adding an identifier to indicate the device has been modified from its original condition, such as a adding a label or other marking to designate the device as reprocessed, refurbished or remanufactured.

After step S6, the operator inspects and tests the newly formed forceps 100 (step S7). Specifically, the operator who remanufactures verifies that the newly formed forceps has the same effectiveness and safety as the original product by various functional tests, such as electrical testing the performance of the conductor layer. There is an advantage that it is easy to verify the performance in Step S7.

After Step S7, the operator sequentially performs a sterilization and storage (Step S8), and shipping (Step S9) of the new forceps 100. In the Step S8, a sterilization treatment using a sterilizing gas such as ethylene oxide gas or propylene oxide gas is applied to the new forceps 100 and the device is stored in a storage container until use.

Steps S1 to S9 described above are executed to achieve reprocessing of the forceps 100.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

1.-20. (canceled)

21. A forceps comprising:
a forceps jaw, the forceps jaw including:
a forceps jaw frame; and
a plurality of standoffs;
a polymer overmold material overmolded on the forceps jaw frame; and
an electrical conductor layer located on a polymer overmold surface of the polymer overmold, the electrical conductor layer including electrically conductive traces extending from a surface of the electrical conductor layer where the plurality of standoffs are located on the polymer overmold surface and separate opposing ones of the electrically conductive traces in a closed position of the forceps jaw.

22. The forceps of claim 21, wherein the polymer overmold includes injected molded silicone.

23. The forceps of claim 21, wherein at least one of the plurality of standoffs includes a ceramic standoff.

24. The forceps of claim 21, wherein each of the plurality of standoffs are unitary with the polymer overmold and project from the polymer overmold surface.

25. The forceps of claim 24, wherein electrically conductive ink is deposited on the polymer overmold surface according to a pattern that creates respective voids of the electrically conductive ink around the plurality of standoffs or around individual ones of the plurality of standoffs.

26. The forceps of claim 24, wherein electrically conductive ink is printed on the polymer overmold surface according to a pattern that creates respective voids of the electrically conductive ink around the plurality of standoffs or around individual ones of the plurality of standoffs.

27. The forceps of claim 21, wherein the electrically conductive layer is formed by additive manufacturing.

28. The forceps of claim 21, further comprising:
a second forceps jaw including a second forceps jaw frame opposing the forceps jaw frame; and
a second polymer overmold located on the second forceps jaw frame and opposing the polymer overmold, the second polymer overmold having a second electrical conductor layer on a surface of the second polymer overmold.

29. A forceps comprising:
a first forceps jaw including a first forceps jaw frame;
a first polymer overmold located on the first forceps jaw frame, the first polymer overmold having a first electrical conductor layer formed on a first polymer overmold surface of the first polymer overmold, the first electrical conductor layer including an electrically conductive ink having a first pattern on the first polymer overmold surface;
a second forceps jaw including a second forceps jaw frame opposing the first forceps jaw frame; and
a second polymer overmold located on the second forceps jaw frame and opposing the first polymer overmold, the second polymer overmold having a second electrical conductor layer on a surface of the second polymer overmold, the second electrical conductor layer including the electrically conductive ink having a second pattern on the second polymer overmold, wherein a first footprint of the first pattern of the first electrical conductor layer is non-overlapping with a second footprint of the second pattern of the second electrical conductor layer.

30. The forceps of claim 29, wherein the first polymer overmold and the second polymer overmold are each injected molded.

31. The forceps of claim 29, wherein the electrically conductive ink is deposited on the first polymer overmold surface in the first pattern and the electrically conductive ink is deposited on the second polymer overmold surface in the second pattern.

32. The forceps of claim 29, wherein the electrically conductive ink is printed on the first polymer overmold surface in the first pattern and the electrically conductive ink is printed on the second polymer overmold surface in the second pattern.

33. The forceps of claim 29, wherein the first pattern is formed adjacent an outer periphery of the first polymer overmold.

34. The forceps of claim 33, wherein the second pattern is formed at an inner portion of the second polymer overmold such that the first pattern alternates with the second pattern.

35. A method of forming forceps, the method comprising:
providing a forceps jaw, the forceps jaw including:
a forceps jaw frame; and
a plurality of standoffs;
forming a polymer overmold on the forceps jaw frame; and
forming an electrically conductive ink layer on a surface of the polymer overmold such that an electrical conductor layer is formed on the surface of the polymer overmold, the electrical conductor layer including electrically conductive traces extending from a surface of the electrical conductor layer where the plurality of standoffs are located on the polymer overmold surface and separate opposing ones of the electrically conductive traces in a closed position of the forceps jaw.

36. The method of claim 35, wherein the polymer overmold is injected molded.

37. The method of claim 35, wherein forming the electrically conductive ink layer comprises depositing electrically conductive ink on the polymer overmold surface according to a pattern that creates respective voids around the plurality of standoffs or around individual ones of the plurality of standoffs.

38. The method of claim 35, wherein forming the electrically conductive ink layer comprises printing the electrically conductive ink layer on the polymer overmold surface according to a pattern that creates respective voids around the plurality of standoffs or around individual ones of the plurality of standoffs.

39. The method of claim 35, wherein the electrically conductive ink is formed on the polymer overmold such that the electrical conductor layer has a first pattern and the method further comprises:
providing a second forceps jaw including a second forceps jaw frame;
forming a second polymer overmold on the second forceps jaw frame;
depositing the electrically conductive ink layer on a surface of the second polymer overmold such that a second electrical conductor layer is formed on the second polymer overmold has a second pattern that alternates with the first pattern.

40. The method of claim 35, wherein each of the plurality of standoffs are unitary with the polymer overmold and project from the polymer overmold surface.
